(19) United States
(12) Patent Application Publication
(10) Pub. No.: US 2021/0030868 A1
(43) Pub. Date: Feb. 4, 2021

(54) FORMULATION OF ANTIBODY BASED DRUGS FOR TREATING LUNG CANCER BY INHALATION

(71) Applicants: Cai Gu Huang, Shrewsbury, MA (US); Hai Long Zhang, Shanghai (CN); Ning He, Shanghai (CN)

(72) Inventors: Cai Gu Huang, Shrewsbury, MA (US); Hai Long Zhang, Shanghai (CN); Ning He, Shanghai (CN)

(73) Assignee: Cai Gu Huang, Shrewsbury, MA (US)

(21) Appl. No.: 16/942,339

(22) Filed: Jul. 29, 2020

Related U.S. Application Data
(60) Provisional application No. 62/879,547, filed on Jul. 29, 2019.

(51) Int. Cl. A61K 39/395; A61K 9/00; A61K 47/22; A61K 47/26; A61K 47/12

(52) U.S. Cl. CPC A61K 39/3955; A61K 9/0078; A61K 2039/544; A61K 47/26; A61K 47/12; A61K 47/22

(57) ABSTRACT

The present invention relates to formulations and method of drug administration useful for treating lung cancer (in particular, non-small lung cancer) by administering a therapeutic antibody drug with a soft mist inhaler or by nebulization.

### PRIORITY STATEMENT

This application claims the benefit of the filing date of U.S. Provisional Patent Application No. 62/879,547, filed on Jul. 29, 2019, which is incorporated herein by reference in its entirety.

### FIELD OF THE INVENTION

The present invention relates to formulations and method of drug administration useful for treating lung cancer (in particular, non-small lung cancer) by administering a therapeutic antibody drug with a soft mist inhaler or by nebulization.

### BACKGROUND OF THE INVENTION

Therapeutic monoclonal antibodies and antibody-based modalities are in development today more than ever before. A faster and more accurate drug discovery process will ensure that the number of candidates coming to the biopharmaceutical pipeline will increase in the future.

Cancer is one of the leading causes of death worldwide. Lung cancer, in particular, is among the top 3 most prevalent cancers, and has a very poor survival rate. As per disease progression SEER (Surveillance, Epidemiology, and End Results database) staging, the five-year relative survival rate for lung cancer is 6% for distant stage, 35% for regional stage, 61% for localized, and 24% for all SEER stages combined. Despite the availability of many cancer drugs it has been difficult and, in the case of some cancer types almost impossible, to improve cure rates or survival. There are many reasons for this lack of success but one reason is the inability to deliver adequate amounts of the drugs to the tumor without causing debilitating and life threatening toxicities in the patient. Indeed, most chemo therapeutic drugs used to treat cancer are highly toxic to both normal and tumor tissues.

Important advancements in the treatment of non-small cell lung cancer (NSCLC) have been achieved over the past two decades, including increased understanding of the disease biology and mechanisms of tumor progression, and advances in early detection and multimodal treatments. The use of small molecule tyrosine kinase inhibitors and immunotherapy has led to unprecedented survival benefits in selected patients. However, the overall cure and survival rates for NSCLC remain low, particularly in metastatic disease. Medications can be taken in a variety of ways, such as by swallowing, by inhalation, by absorption through the skin, or by intravenous injection. Each method has advantages and disadvantages, and not all methods can be used for every medication. Improving current delivery methods or designing new ones can enhance the efficacy and use of existing medications to expand the clinical benefit to a broader patient population and to improve outcomes in NSCLC.

As part of the Biologics Price Competition and Innovation Act (BPCIA), a biological drug product (produced in or derived from living organisms) may be demonstrated to be “biosimilar” if data show that, among other things, the product is “highly similar” to an already-approved biological product. The biosimilar product should retain at least the biologic function and treatment efficacy of the U.S. Food and Drug Agency-approved biological product. The biosimilar product can be formulated differently, however, from the approved biological product. The different formulation can provide improved stability and shelf storage of the biologic drug product, and can also improve the efficacy in treating a particular disease or condition. The different formulation can also improve other aspects of administration, including a reduction in patient discomfort or other unwanted effects that a patient may experience upon administration of the approved biological product. Antibody molecules can be produced as a biosimilar, and reformulated accordingly. There remains a need in the art for high quality antibody formulation, method of administration, and use thereof.

Currently, systemic intravenous administration of lung cancer drugs can only deliver about 9-10% of the drug to the tumor site in the lung, requiring a high dosage of cancer medicine. The IV administration route of a drug exposes the entire body to the drug. Although doses are selected that destroy tumor cells, these doses also destroy normal cells. As a result, the patient usually experiences severe toxic side effects. For example, severe myelosuppression may result which compromises the ability of the patient to resist infection and allows spread of the tumor. There are other life-threatening effects such as hepatotoxicity, renal toxicity, pulmonary toxicity, cardiotoxicity, neurotoxicity, and gastrointestinal toxicity caused by anticancer drugs. Moreover, a significant amount of drug remains in the blood circulatory system and causing severe side effects, as well as adverse effects. Moreover, it is important to note that these toxicities are not associated to the same extent with all anticancer drugs, but are all due to systemic delivery of the drug.

Differences in mechanisms of action and pharmacokinetic properties of the various anticancer drugs against different tumor types, which exhibit various biological behaviors, determine, in part, their efficacy.

The concept of local drug delivery is proposed as a method for delivering high drug concentrations to a target site while preventing exposure of vital organs to toxic drug concentrations via systemic circulation. In this way, systemic side effects are minimized. The respiratory system has a large surface area, thin alveolar epithelium, rapid absorption, lack of first-pass metabolism, high bioavailability, and the capacity to absorb large quantities of drug, making it an optimal route of drug administration. (Labiris and Dolovich 2003)

In order to achieve localized delivery of an active substances into the lung, it is clinically advantageous to use a liquid formulation of the active substance administered using a suitable inhaler. Moreover, it is very important to increase lung deposition of a drug delivered by inhalation using soft mist inhalation or nebulizer inhalation. Therefore, there is a need to improve drug delivery of effective cancer medicines by increasing lung deposition of the cancer medicine. A soft mist inhalation device or other nebulization devices can significantly increase the lung deposition of liquid drug formulations.

U.S. Pat. No. 6,471,943B1 suggests that, highly toxic, vesicant and previously unknown nonvesicant, antineoplastic drugs can be effectively delivered to a patient in need of treatment for neoplasms or cancers by inhalation. This route is particularly effective for treatment of neoplasms or cancers of the pulmonary system because the highly toxic drugs are delivered directly to the site where they are needed, providing regional doses much higher than can be achieved by conventional IV delivery.

Konstantinos et. al. recently studied the three immunotherapeutic drugs nivolumab, ipilimumab, and pembrolizumab, which can be produced as an aerosol from their current form in water as a solvent using a jet-nebulizer and residual cup. (Sapalidis, Zarogoulidis et al. 2018)

The main objective when formulating a therapeutic monoclonal antibody solution for administration using an inhaler to treat NSCLC is to increase the efficacy of the therapeutic monoclonal antibody and to reduce the dosage and side effects caused during the IV infusion. The general disadvantage of therapeutic monoclonal antibody IV infusion is its route of administration, high dose, and stability. Once the infusion solution is prepared it has to be administered through an intravenous line as soon as possible as it can be stored for only 24 hours in a refrigerator at 2° C. to 8° C. or 8 hours at room temperature.

### SUMMARY OF THE INVENTION

The present invention is directed to a novel therapeutic strategy for the treatment of metastatic NSCLC through a soft mist inhaler or nebulizer. Therapeutic monoclonal antibodies for metastatic NSCLC are formulated to form an aerosol using a soft mist inhaler. The aerosolized therapeutic monoclonal antibodies are locally delivered to a lung tumor by inhalation. The local delivery of the therapeutic monoclonal antibody is aimed to increase efficacy for treating metastatic NSCLC by increasing lung deposition. This therapeutic strategy reduces the side effects of the drug because very low concentrations of the antibody are absorbed through the alveoli and enter the blood circulatory system. The local delivery of a therapeutic monoclonal antibody through inhalation reduces the dosage of the therapeutic antibody compare to systematic IV administration and, thus, reduces the toxicity.

### BRIEF DESCRIPTION OF THE DRAWINGS

FIG. 1 shows a longitudinal section through an atomizer in the stressed state.

FIG. 2 shows the counter element of an atomizer.

The use of identical or similar reference numerals in different figures denotes identical or similar features.

### DETAILED DESCRIPTION OF THE INVENTION

The technical and nontechnical terminology used herein is for the purpose of describing particular embodiments only, and is not intended to be limiting of the invention. As used herein, the term “and/or” includes any and all combinations of one or more of the associated listed items. As used herein, the singular forms “a”, “an”, and “the” are intended to include the plural forms as well as the singular forms, unless the context clearly indicates otherwise. It will be further understood that the term “comprises” when used in this specification, specifies the presence of the stated features, steps, operations, elements, and/or components, but do not preclude the presence or addition of one or more other features, steps, operations, elements, components and/or groups thereof.

Unless otherwise defined, all terms (including technical and scientific terms) used herein have the same meaning as commonly understood by one having ordinary skill in the art to which the invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used herein the respiratory tract includes the oral and nasal-pharyngeal, tracheobronchial, and pulmonary regions. The pulmonary region is defined to include the upper and lower bronchi, bronchioles, terminal bronchioles, respiratory bronchioles, and alveoli.

In describing the invention, it will be understood that a number of formulations and steps are disclosed. Each of these has individual benefits and each can also be used in conjugation with one or more, or in some cases all, of the other disclosed techniques. Accordingly, for the sake of clarity, this description will refrain from repeating every possible combination of the individual steps in an unnecessary fashion. Nevertheless, the specification and claims should be read with an understanding that such combinations are entirely within the scope of invention and the claims.

The present disclosure is to be considered as an exemplification of the invention, and is not intended to limit the invention to the specific embodiments illustrated by the figures or description below.

The present invention describes a pharmaceutical formulation of an active therapeutic monoclonal antibody with other excipients that can be administered by soft mist inhalation or nebulizer inhalation for the treatment of NSCLC. The invented formulations for the soft mist inhaler should meet standard quality guidelines. Therefore, one aim of the current invention is to provide a stable formulation containing a therapeutic monoclonal antibody in functional form with inactive ingredients in a solution, which meet the standard delivered dosage requirements needed to achieve optimum nebulization of the solution using the soft mist inhaler. It is most important to formulate the pharmaceutically active formulation as the most stable solution to keep the active ingredient functional for the labeled dosage. Another aspect is to provide a propellant-free suspension containing a therapeutic monoclonal antibody and excipients, which is nebulized under pressure using a soft mist or nebulization inhaler device. The amount of the composition delivered by the aerosol is reproducibly produced within a specified range.

The present invention relates to an inhalable NSCLC therapeutic monoclonal antibody formulation containing the anticancer monoclonal antibody as a major active molecule in a mixture of sodium chloride, sodium citrate dihydrate, mannitol, pentetic acid, polysorbate 80 as inactive ingredients. Preferably, the mixture is administered as an aerosol formed from a soft mist inhaler or nebulizer. The pharmaceutical formulations disclosed in the current invention are especially suitable for soft mist inhalation or nebulization inhalation, which have much better lung depositions, typically up to 55-60%, compared to the IV infusion. Furthermore, liquid inhalation formulations of therapeutic monoclonal antibodies have other advantages compared to the administration of therapeutic monoclonal antibodies administered through an IV line, particularly for treating NSCLC.

The soft mist inhalers nebulize a small amount of a liquid formulation containing the required dosage of the therapeutic monoclonal antibody within a few seconds into an aerosol that is suitable for therapeutic inhalation. The soft mist inhaler is particularly suitable for the liquid formulation disclosed in the current invention. A parameter of the aerosol, which is indicative of the aerosol quality, is the so-called inhalable proportion, which is defined herein as the proportion of the mist droplets with a measured median aero-dynamic diameter (MMAD) of less than about 10 μm. The inhalable proportion can be measured using an “Andersen Impactor”. For good protein absorption it is important to not only achieve aerosolization without any substantial loss of activity but to also generate an aerosol with a good inhalable proportion. Aerosols with an MMAD of less than about 10 μm are significantly better suited to reaching the alveoli, where their chances of being absorbed are significantly greater. The effectiveness of a soft mist inhaler (SMI) device can also be tested in an in vivo system. As an example of an in vivo test system, a protein-containing mist can be administered to a dog through a tracheal tube. Blood samples are taken at suitable time intervals and the protein level in the plasma are then measured by immunological or biological methods.

The invention also relates to aerosol preparations in the form of an aqueous solution that contain as an active substance a biologically active macromolecule, particularly a therapeutic antibody, in an amount of between about 1 mg/ml and about 100 mg/ml, preferably between about 10 mg/ml and about 100 mg/ml.

Preferably, the therapeutic monoclonal antibody for treating metastatic NSCLC with the current invention is Nivolumab, Ipilibumab, Atezolizumab, Pembrolizumab, Durvalumab, Avastin or combinations thereof.

The pharmaceutical formulation according to the invention may be formulated using one or more physiologically acceptable carriers comprising excipients and auxiliaries known in the art. Preferably, said excipients and auxiliaries are selected from L-Histidine (molecular formula is C₆H₉N₃O₂, molecular weight is: 155.15 g/mol, the IUPAC name is (2S)-2-amino-3-(1H-imidazol-5-yl) propanoic acid); Sodium citrate dehydrate (molecular formula is C₆H₉Na₃O₉, molecular weight is 294.098 g/mol, the IUPAC name is trisodium 2-hydroxypropane-1,2,3-tricarboxylate dehydrate); Sodium chloride (molecular weight is 58.44 g/mol and the IUPAC name is sodium chloride); Mannitol (molecular formula is C₆H₁₄O₆, molecular weight is 182.172 g/mol, the IUPAC name is (2R,3R,4R,5R)-hexane-1,2,3,4,5,6-hexol); Pentetic acid (molecular formula is C₁₄H₂₃N₃O₁₀, molecular weight is 393.349 g/mol, the IUPAC name is 2-[bis[2-[bis(carboxymethyl) amino]ethyl]amino] acetic acid); Polysorbate 80 (molecular formula is C₃₂H₆₀O₁₀, molecular weight is 604.822 g/mol, the IUPAC name is 2-[2-[3,5-bis(2-hydroxyethoxy)oxolan-2-yl]-2-(2-hydroxyethoxy)ethoxy]ethyl (E)-octadec-9-enoate); α,α-Trehalose dehydrate (molecular formula is C₁₂H₂₆O₁₃, molecular weight is 378.33 g/mol, the IUPAC name is (2R,3S,4S,5R,6R)-2-(hydroxymethyl)-6-[(2R,3R,4S,5S,6R)-3,4,5-trihydroxy-6-(hydroxymethyl)oxan-2-yl]oxyoxane-3,4,5-triol; dehydrate); and Sucrose (molecular formula is C₁₂H₂₂O₁₁, molecular weight is 342.3 g/mol, the IUPAC Name is (2R,3R,4S,5S,6R)-2-[(2S,3S,4S,5R)-3,4-dihydroxy-2,5-bis(hydroxymethyl)oxolan-2-yl]oxy-6-(hydroxymethyl)oxane-3,4,5-triol).

The formulation of the present invention may include chelating agents, preservatives, antioxidants, processing aids, and other additives.

To produce the propellant-free aerosols according to the invention, the pharmaceutical formulation containing the antibody is preferably administered with a soft mist inhalation device.

A typical device for the propellant-free administration of a metered amount of a liquid pharmaceutical composition for soft mist inhalation is described in detail in, for example, US20190030268 “inhalation atomizer comprising a blocking function and a counter”.

The pharmaceutical solution in the nebulizer is converted into aerosol destined for the lungs. The pharmaceutical solution is sprayed with the nebulizer by high pressure.

The inhalable device can be carried anywhere by the patient, since its cylindrical shape and handy size is less than 8 cm to 18 cm long, and 2.5 cm to 5 cm wide. The nebulizer sprays out a defined volume of the pharmaceutical formulation through small nozzles at high pressures, so as to produce an inhalable aerosol.

The preferred atomizer comprises an atomizer 1, a fluid 2, a vessel 3, a fluid compartment 4, a pressure generator 5, a holder 6, a drive spring 7, a delivering tube 9, a non-return valve 10, pressure room 11, a nozzle 12, a mouthpiece 13, an aerosol 14, an air inlet 15, an upper shell 16, an inside part 17.

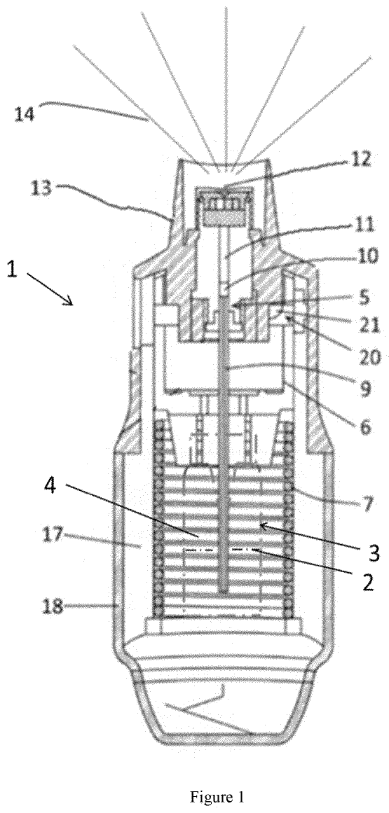

The inhalation atomizer 1 comprising the block function and the counter described above for spraying a medicament fluid 2 is depicted in the FIG. 1 in a stressed state. The atomizer 1 comprising the block function and the counter described above is preferably a portable inhaler and requires no propellant gas.

FIG. 1 shows a longitudinal section through the atomizer in a stressed state.

For the typical atomizer 1 comprising the block function and the counter described above, an aerosol 14, which can be inhaled by a patient, is generated through atomization of the fluid 2, which is preferably formulated as a medicament liquid. The medicament is typically administered at least once a day, more specifically multiple times a day, preferably at predestined time gaps, according to how serious the illness affects the patient.

A preferred atomizer 1 comprising the block function and the counter described above has a substitutable and insertable vessel 3, which contains the medicament fluid 2. Therefore, a reservoir for holding the fluid 2 is formed in the vessel 3. Specifically, the medicament fluid 2 is located in the fluid compartment 4 formed by a collapsible bag in the vessel 3.

A preferred adequate amount of fluid 2 in the vessel 3 of the inhalation atomizer 1 comprising the block function and the counter described above is an amount sufficient to provide up to 200 doses. A classical vessel 3 has a volume of about 2 to about 10 ml. A pressure generator 5 in the atomizer 1 is used to deliver and atomize the fluid 2, specifically in a predestined dosage amount. Therefore, the fluid 2 can be released and sprayed in individual doses, preferably from about 5 to about 30 microliter.

The typical atomizer 1 comprising the block function and the counter described above preferably has a pressure generator 5 and a holder 6, a drive spring 7, a delivering tube 9, a non-return valve 10, a pressure room 11, and a nozzle 12 in the area of a mouthpiece 13. The vessel 3 is latched by the holder 6 in the atomizer 1 so that the delivering tube 9 is plunged into the vessel 3. The vessel 3 can be separated from the atomizer 1 for substitution.

For the atomizer 1 comprising block function and the counter described above, when drive spring 7 is stressed in an axial direction, the delivering tube 9 and the vessel 3, along with the holder 6, will be shifted downwards. Then the fluid 2 will be sucked into the pressure room 11 through delivering tube 9 and non-return valve 10.

For the inhalation atomizer 1 comprising the block function and the counter described above, after the holder 6 is released, the stress is eased. During this process, the delivering tube 9 and closed non-return valve 10 are shifted back upward by releasing the drive spring 7. Consequently, the fluid 2 is under the pressure in the pressure room 11. The fluid 2 is then pushed through the nozzle 12 and atomized into an aerosol 14 by the pressure. A patient can inhale the aerosol 14 through the mouthpiece 13, while the air is sucked into the mouthpiece 13 through air inlets 15.

The inhalation atomizer 1 comprising the block function and the counter described above has an upper shell 16 and an inside part 17, which can be rotated relative to the upper shell 16. A lower shell 18 is manually operable to attach onto the inside part 17. The lower shell 18 can be separated from the atomizer 1 so that the vessel 3 can be substituted and inserted.

The inhalation atomizer 1 comprising the block function and the counter described above preferably has the lower shell 18, which carries the inside part 17, rotatable relative to the upper shell 16. As a result of rotation and engagement between the upper unit 17 and the holder 6, through gear 20, the holder 6 axially moves the counter so that the drive spring 7 is stressed.

In the stressed state, the vessel 3 is shifted downwards to reach a final position, which is depicted in the FIG. 1. The drive spring 7 is stressed in this final position and the holder 6 is clasped. Therefore, the vessel 3 and the delivering tube 9 are prevented from moving upwards so that the drive spring 7 is stopped from easing.

The typical atomizing process occurs after releasing the holder 6. The vessel 3, the delivering tube 9 and the holder 6 are shifted back by the drive spring 7 to the starting position. This is referred to herein as major shifting. When the major shifting occurs, the non-return valve 10 is closed and the fluid 2 is under pressure in the pressure room 11, and then the fluid 2 is pushed out and atomized by the pressure.

The inhalation atomizer 1 comprising the block function and the counter described above preferably has a clamping function. During clamping, the vessel 3 preferably performs a lifting shift for withdrawal of the fluid 2 during the atomizing process. The gear 20 has sliding surfaces 21 on the upper shell 16 and/or on the holder 6, which makes holder 6 axially move when the holder 6 is rotated relative to the upper shell 16.

The holder 6 is not blocked for too long and can carry on the major shifting. Therefore, the fluid 2 is pushed out and atomized.

In this clamping function, when the holder 6 is in the clamping position, the sliding surfaces 21 move out of engagement. Then the gear 20 releases the holder 6 to shift axially in the opposite direction.

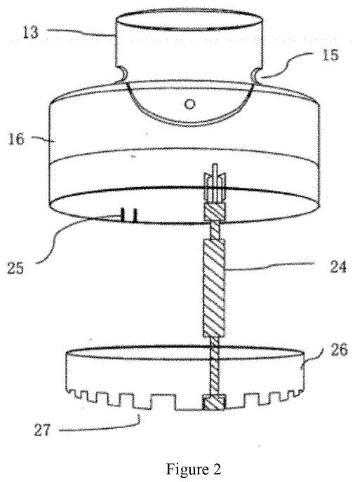

The atomizer 1 preferably includes a counter element showed in FIG. 2. The counter element has a worm 24 and a counter ring 26. The counter ring 26 is preferably circular and has dentate part at the bottom. The worm 24 has upper and lower end gears. The upper end gear contacts with the upper shell 16. The upper shell 16 has an inside bulge 25. When the atomizer 1 is employed, the upper shell 16 rotates; and when the bulge 25 passes through the upper end gear of the worm 24, the worm 24 is driven to rotate. The rotation of the worm 24 drives the rotation of the counter ring 26 through the lower end gear to result in the counting effect.

The locking mechanism is realized mainly by two protrusions. Protrusion A is located on the outer wall of the lower unit of the inside part. Protrusion B is located on the inner wall of counter. The lower unit of the inside part is nested in the counter. The counter rotates relative to the lower unit of the inside part. Because of the rotation of the counter, the number displayed on the counter changes as the actuation number increases, and can be observed by the patient. After each actuation, the number displayed on the counter changes. Once the predetermined number of actuations is achieved, Protrusion A and Protrusion B will encounter with each other and hence the counter will be prevented from further rotation. Therefore, the atomizer is blocked and stopped from further use. The number of actuations of the device is counted by the counter.

The nebulizer described above is suitable for nebulizing the aerosol preparations according to the invention to form an aerosol suitable for inhalation. Nevertheless, the formulation according to the invention can also be nebulized using other inhalers apart from those described above, such as an ultrasonic vibrating mesh nebulizer, and a compressed air nebulizer.

A typical ultrasonic vibrating mesh nebulizer is composed of a liquid reservoir with a piezo mesh disk mounted on one side of it and a piezo mesh driver circuit board with batteries. The piezo mesh disk consists of a stainless steel plate that has been perforated with thousands of precision-formed, laser-drilled holes, and surrounded by a piezoelectric material. The piezoelectric material vibrates at a very high rate of speed when it is driven by an analog signal of specific voltage, frequency, and waveform that is generated by the driver board. As a result of the rapid vibration, solution is drawn through the holes to form droplets of consistent size that are delivered at a low velocity for inhalation directly into the lungs.

With a typical compressed air nebulizer, an aerosol is generated by passing air flow in a nebulizer bowl. This forms a low-pressure zone that pulls up droplets through a feed tube from a solution or suspension of drug in the nebulizer bowl, which in turn creates a stream of atomized droplets, which flow to the mouthpiece. Higher air flows lead to a decrease in particle size and an increase in output. A baffle in the nebulizer bowl is impacted by larger particles, retaining them and returning them to the solution in the nebulizer bowl to be re-atomized. There is considerable variation in the performance of nebulizers. In addition, nebulizers require a source of compressed air.

### EXAMPLES

Formulation Ingredients:

Pembrolizumab, Nivolumab, Atezolizumab, Durvalumab, Avastin, L-Histidine, L-Histidine hydrochloride monohydrate, polysorbate 80, polysorbate 20, sodium chloride, Sodium citrate dihydrate, sodium phosphate monobasic, sodium phosphate dibasic, mannitol, pentetic acid, α, α-trehalose dihydrate, and sucrose.

### Example 1

Preparation of an aqueous solution containing Pembrolizumab as an active ingredient for soft mist inhalation is as follows: 5 ml of a Pembrolizumab (10 mg/ml, and 20 mg/ml) solution was prepared by dissolving L-Histidine, Polysorbate 80, and sucrose in 4 ml of sterile water as described in table 1, and the solution was adjusted to the target pH with hydrochloric acid. Finally sterile water was added to provide a final volume of 5 ml.

TABLE 1
Formulation ingredient contents of sample I and sample II.

| | | |
|---|---|---|
| Ingredients | Sample I | Sample II |

| | | | | |
|---|---|---|---|---|
| Pembrolizumab | 50 | mg | 100 | mg |
| L-histidine | 3.1 | mg | 6.2 | mg |
| Polysorbate 80 | 0.4 | mg | 0.8 | mg |
| Sucrose | 140 | mg | 280 | mg |

| | | |
|---|---|---|
| pH | 5.5 | 5.5 |

| | | | | |
|---|---|---|---|---|
| Sterile water | 5 | ml | 5 | ml |

### Example 2

The preparation of Atezolizumab solution for soft mist inhalation is as follows: 5 ml of an Atezolizumab (30 mg/ml or 60 mg/ml) solution was prepared by adding and dissolving L-histidine, Polysorbate 20, and Sucrose in water as described in table 2, and the solution was adjusted to the target pH with glacial acetic acid. Finally, sterile water was added to make a final volume of 5 ml.

TABLE 2
Formulation ingredient contents of sample I, and sample II.

| | | |
|---|---|---|
| Ingredients | Sample I | Sample II |

| | | | | |
|---|---|---|---|---|
| Atezolizumab | 150 | mg | 300 | mg |
| L-histidine | 155 | mg | 310 | mg |
| Polysorbate 20 | 20 | mg | 40 | mg |
| Sucrose | 2054 | mg | 4108 | mg |
| Glacial acetic acid | 41.25 | mg | 82.8 | mg |

| | | |
|---|---|---|
| pH | 5.8 | 5.8 |

| | | | | |
|---|---|---|---|---|
| Sterile water | 5 | ml | 5 | ml |

### Example 3

Formulation of an aqueous solution containing Nivolumab for soft mist inhalation is prepared as follows: a 5 ml of Nivolumab (5 mg/ml or 10 mg/ml) solution was prepared by adding and dissolving mannitol, sodium chloride, polysorbate 80, sodium citrate dihydrate in 4 ml of sterile water as described in table 3. The solution was adjusted to the target pH with pentetic acid. Finally, sterile water was added to make final volume to 5 ml.

TABLE 3
Formulation ingredient contents of sample I, and sample II.

| | | |
|---|---|---|
| Ingredients | Sample I | Sample II |

| | | | | |
|---|---|---|---|---|
| Nivolumab | 25 | mg | 50 | mg |
| Mannitol | 75 | mg | 150 | mg |
| Sodium chloride | 7.3 | mg | 14.6 | mg |
| Polysorbate 80 | 0.5 | mg | 1 | mg |
| Sodium citrate dihydrate | 14.7 | mg | 29.4 | mg |
| Pentetic acid | 0.02 | mg | 0.04 | mg |

| | | |
|---|---|---|
| pH | 6 | 6 |

| | | | | |
|---|---|---|---|---|
| Sterile water | 5 | ml | 5 | ml |

### Example 4

The preparation of Durvalumab solution for soft mist inhalation is as follows: 5 ml of a Durvalumab (25 mg/ml or 50 mg/ml) solution was prepared by adding and dissolving L-histidine, L-histidine hydrochloride monohydrate, α, α,-trehalose dihydrate, and polysorbate 80 in 4 ml of sterile water as described in table 4. The solution was then adjusted to the target pH with hydrochloric acid. Finally, sterile water was added to make a final volume 5 ml.

TABLE 4
Formulation ingredient contents of sample I, and sample II.

| | | |
|---|---|---|
| Ingredients | Sample I | Sample II |

| | | | | |
|---|---|---|---|---|
| Durvalumab | 125 | mg | 250 | mg |
| L-histidine | 5 | mg | 10 | mg |
| L-histidine hydrochloride | 6.75 | mg | 13.5 | mg |
| monohydrate | | | | |
| α,α,-trehalose dihydrate | 260 | mg | 520 | mg |
| Polysorbate 80 | 0.5 | mg | 1 | mg |

| | | |
|---|---|---|
| pH | 6 | 6 |

| | | | | |
|---|---|---|---|---|
| Sterile water | 5 | ml | 5 | ml |

### Example 5

The preparation of Bevacizumab solution for soft mist inhalation is as follows: 5 ml of an Avastin (15 mg/ml or 25 mg/ml) solution was prepared by adding α,α-trehalose dihydrate, sodium phosphate (monobasic), sodium phosphate (dibasic), and polysorbate 80 in 4 ml sterile water as described in table 5. The solution was adjusted to the target pH with hydrochloric acid. Finally, sterile water was added to make a final volume 5 ml.

TABLE 5
Formulation ingredient contents of sample I, and sample II.

| | | |
|---|---|---|
| Ingredients | Sample I | Sample II |

| | | | | |
|---|---|---|---|---|
| Bevacizumab (Avastin) | 75 | mg | 125 | mg |
| α,α-trehalose dihydrate | 180 | mg | 300 | mg |
| Sodium phosphate (monobasic, | 17.4 | mg | 29 | mg |
| monohydrate) | | | | |
| Sodium phosphate (dibasic, | 3.6 | mg | 6 | mg |
| anhydrous) | | | | |
| Polysorbate 20 | 1.2 | mg | 2 | mg |

| | | |
|---|---|---|
| pH | 5.8 | 5.8 |

| | | | | |
|---|---|---|---|---|
| Sterile water | 5 | ml | 5 | ml |

What is claimed is:

1. A liquid, propellant-free pharmaceutical preparation comprising: (a) an active substance; (b) a solvent; and (c) a pharmacologically acceptable excipient.

2. The pharmaceutical preparation according to claim 1, wherein the active substance is selected from the group consisting of Atezolizumab, Nivolumab, Pembrolizumab, Durvalumab, Avastin, and any combination thereof.

3. The pharmaceutical preparation according to claim 1, wherein active substances is present in an amount of from about 1 mg/ml to about 100 mg/ml.

4. The pharmaceutical preparation according to claim 1, wherein the pharmacologically acceptable excipient is selected from the group consisting of L-Histidine, L-Histidine hydrochloride monohydrate, sodium citrate dihydrate, polysorbate 80, polysorbate 20, sodium chloride, sodium phosphate, mannitol, pentetic acid, α, α-trehalose dehydrate, sucrose, and any combination thereof.

5. The pharmaceutical preparation according to claim 1, comprising about 1 mg/ml to about 25 mg/ml Pembrolizumab, about 1 mM to about 10 mM of L-histidine, about 50 mM to about 200 mM sucrose, and about 0.05 mM to about 0.15 mM polysorbate 80, wherein the pharmaceutical preparation has a pH of about 5.5 to about 5.7, and the pharmaceutical preparation is stable for at least 12 months when stored under refrigerated conditions at 2° C. to 8° C.

6. The pharmaceutical preparation according to claim 1, comprising about 10 mg/ml to about 65 mg/ml of Atezolizumab, about 50 mM to about 450 mM of L-histidine, about 500 mM to about 2450 mM sucrose, about 1 mM to about 10 mM polysorbate 20, and about 60 mM to about 300 mM of glacial acetic acid, wherein the pharmaceutical preparation has a pH of about 5.8 to about 6, and the pharmaceutical preparation is stable for at least 12 months when stored under refrigerated conditions at 2° C. to 8° C.

7. The pharmaceutical preparation according to claim 1, comprising about 5 mg/ml to 10 mg/ml of Nivolumab, about 0.05 mM to about 200 mM of polysorbate 80, about 10 mM to about 30 mM sodium citrate dihydrate, about 20 mM to about 60 sodium chloride, about 50 mM to about 200 mM of mannitol, and about 0.005 mM to about 0.025 mM pentetic acid, wherein the pharmaceutical preparation has a pH of about 6, and the pharmaceutical preparation is stable for at least 12 months when stored under refrigerated conditions at 2° C. to 8° C.

8. The pharmaceutical preparation according to claim 1, comprising about 10 mg/ml to 55 mg/ml of Durvalumab, about 1 mM to about 15 mM of L-histidine, about 0.03 mM to about 0.2 mM polysorbate 80, about 1 mM to about 15 L-histidine hydrochloride monohydrate, and about 50 mM to about 300 mM of trehalose dihydrate, wherein the pharmaceutical preparation has a pH of about 6, and the pharmaceutical preparation is stable for at least 12 months when stored under refrigerated conditions at 2° C. to 8° C.

9. The pharmaceutical preparation according to claim 1, comprising about 10 mg/ml to 25 mg/ml of Bevacizumab, about 0.15 mM to about 0.35 mM polysorbate 20, about 15 mM to about 55 monobasic sodium phosphate monohydrate, about 1 mM to about 10 mM of dibasic sodium phosphate dihydrate, and about 50 mM to about 200 mM of trehalose dehydrate, wherein the pharmaceutical preparation has a pH of about 6, and the pharmaceutical preparation is stable for at least 12 months when stored under refrigerated conditions at 2° C. to 8° C.

10. A method of administering the pharmaceutical preparation according to claim 1, comprising nebulizing the pharmaceutical preparation in an inhaler according to FIG. 1.

11. A method of treating lung cancer in a patient, comprising administering to the patient the pharmaceutical preparation according to claim 1.

12. The method according to claim 11, further comprising nebulizing the pharmaceutical preparation in an inhaler.

13. The pharmaceutical preparation according to claim 1, comprising a combination of Pembrolizumab and Avastin as the active ingredients.

14. The pharmaceutical preparation according to claim 1, comprising a combination of Atezolizumab and Avastin as the active ingredients.

15. The pharmaceutical preparation according to claim 1, comprising a combination of Durvalumab and Avastin as the active ingredients.

16. The pharmaceutical preparation according to claim 1, comprising a combination of Nivolumab and Avastin as the active ingredients.

17. A method of administering the pharmaceutical formulation of claim 5 comprising producing an aerosol of the pharmaceutical formulation with a nebulizer or a soft mist inhaler.

18. A method of administering the pharmaceutical formulation of claim 6 comprising producing an aerosol of the pharmaceutical formulation with a nebulizer or soft mist inhaler.

19. A method of administering the pharmaceutical formulation of claim 7 comprising producing an aerosol of the pharmaceutical formulation with a nebulizer or soft mist inhaler.

20. A method of administering the pharmaceutical formulation of claim 8 comprising producing an aerosol of the pharmaceutical formulation with a nebulizer or soft mist inhaler.

21. A method of administering the pharmaceutical formulation of claim 9 comprising producing an aerosol of the pharmaceutical formulation with a nebulizer or soft mist inhaler.

22. The process of claim 10, wherein the nebulizing provides aerosol particles having an average size of less than about 10 μm.

23. The process of claim 10, wherein the nebulizing provides aerosol particles having an average size of less than about 5 μm.
